(19) United States
(12) Patent Application Publication
(10) Pub. No.: US 2007/0185406 A1
(43) Pub. Date: Aug. 9, 2007

(54) SYSTEMS AND METHODS FOR PROCESSING PULMONARY FUNCTION DATA

(76) Inventor: Michael David Goldman, Los Angeles, CA (US)

Correspondence Address: WINSTON & STRAWN LLP;PATENT DEPARTMENT, 1700 K STREET, N.W., WASHINGTON, DC, 20006, US

(21) Appl. No.: 11/671,239

(22) Filed: Feb. 5, 2007

Related U.S. Application Data
(60) Provisional application No. 60/771,406, filed on Feb. 7, 2006.

(51) Int. Cl. A61B 5/08

(52) U.S. Cl. 600/533; 600/538; 600/529

(57) ABSTRACT

The invention provides improved methods for analysis of data obtained from certain pulmonary testing procedures, in particular from whole body plethysmography or from the forced oscillation technique. The improved computer-implemented methods automatically recognize data that has been distored by patient behaviors during testing. This invention also provides computer systems that interface to devices that perform whole body plethysmography and/or the forced oscillation technique and automatically execute the methods of this invention. This invention also provides for distribution of software that causes computer systems to perform the methods of this invention.

This application claims the benefit of U.S. provisional application no. 60/771,406 filed Feb. 7, 2006.

### FIELD OF THE INVENTION

The invention relates to analysis of data obtained from certain pulmonary testing procedures, and in particular to improved processing methods for data obtained from whole body plethysmographic techniques and/or the respiratory airflow perturbation techniques.

### BACKGROUND OF THE INVENTION

Whole body plethysmography (“WBP”) and the respiratory airflow perturbation techniques (“RAFD”), are useful methods of measuring aspects of lung function in health and disease. See, e.g., Lung Function Testing, Eds R Gosselink, H Stam. European Respiratory Society Journals, Ltd. Sheffield, UK, 2005, Lung Function Testing, Goldman et al, Chapter 2. Whole-body plethysmography, Smith et al, Chapter 5. Forced oscillation technique and impulse oscillometry.

Whole Body Plethysmographic Techniques

In WBP, a subject sits in a rigid chamber comparable in size and shape to an enclosed telephone booth and breathes through a pneumotachograph. For certain measurements, a shutter in the mouthpiece tubing attached to the pneumotachograph is momentarily closed. Pressure transducers measure the pressure drop across the pneumotachograph (from which subject air flows are determined), plethysmographic pressure (with respect to outside air pressure from which changes in subject's thoracic gas volume [TGV] are determined), and mouth pressure at the airway opening. In the constant-volume or variable-pressure plethysmograph, the subject breathes air from inside the WBP so that changes in subject volume reflect compressive and decompressive changes in total respiratory air volumes. In the constant-pressure or volume-displacement plethysmograph, the subject breathes air from outside the WBP so that changes in subject volume reflect total changes in total lung volume inclusive of respired air volumes and any changes associated with compression or decompression.

To appreciate operation of the constant-pressure plethysmograph, it should be understood that for the following reasons changes in alveolar pressure (“PA”) can be inferred from changes in plethysmograph pressure which in turn reflect changes in net subject volumes (the “shift volume” denoted “ΔV”). The link (or ‘amplification factor’) between PA and ΔV is gas resident in the lung during normal breathing. The pressure in this gas directly reflects PA, and therefore increases during expiration and decreases during inspiration; these pressure changes cause this resident gas to contract and expand thereby changing (“ΔV”) net subject volume. As explained, ΔV is derived from measured changes in plethysmographic pressure.

Accordingly, total gas volume (“TGV”) in the lung can be measured, and the relation between PA and ΔV can be calibrated, by having the subject make breathing efforts against a closed shutter. Under these conditions, ΔV is proportional to TGV while PA is closely related to changes in mouth pressure ΔPM. Subsequently, specific airway resistance (“sRAW”), which is the ratio of airflow (V′) into and out of the lung divided by the change in plethysmographic pressure (reflected by ΔV) can be determined by having the subject breath freely through the pneumotachograph while recording ΔV. sRAW is then V′ measured by the pneumotachograph divided by ΔV. Airway resistance (“RAW”) is subject sRAW normalized by subject TGV. In fact, it has long been clinically appreciated that whole body plethysmographic measurements of RAW and sRAW (and also TGV and ΔV) are considered the “gold standard” for assessing airway function. Such assessments are important in recognizing and treating lung disease.

sRAW measurements are usually displayed as a loop (“sRAW loop”) on a two dimensional graph where mouth air flow recorded by the pneumotachograph is along one axis and ΔV shift volume produced by thoracic compression and decompression is along another axis. In normal lungs, sRAW loops are usually a linear, narrow, oval loop. But in the presence of lung pathology, sRAW loops become complex and nonlinear due at least to the contributions of dynamic compression of intra-thoracic airways and compression of non-ventilated lung.

Attempts are known in the art to represent the complex, nonlinear sRAW loops occurring in lung pathologies by one or two derived numerical parameters including, for example, the total specific resistance (“sRTOT”) and the effective specific resistance effective specific resistance (“sREFF”). sRTOT is the slope of a straight line drawn between maximal inspiratory and maximal expiratory shift volume points of the sRAW loop. See, e.g., Islam et al, 1974, Diagnostic value of ‘closing volume’ in comparison to ‘airway resistance/lung volume plot’; Respiration 31:449-458. Although sensitive to peripheral airway obstruction, this measure cannot reliably represent the full sRAW loop. It can also can be more variable from test to test. Both problems arise because it is a quotient of differences of values of only two extreme points of the sRAW loop.

sREFF is calculated by computer from multiple integrals of WBP measurement data that arises from one or more respiratory cycles. See, e.g., Matthys et al., 1975, Comparative Measurements of Airway Resistance; Respiration 32 :121-134; Jaeger et al., 1954, Measurement of airway resistance with a volume displacement body plethysmograph; J Appl. Physiol. 19: 813-820. First, moment-by-moment lung volumes are determined by integrating measured airflows, and are used to parameterize airflows and shift volumes. Thereby, two loops are formed, a first loop of airflow versus integrated airflow and a second loop of shift volume versus integrated airflow. Next, volume-weighted-average airflow (so-called “effective airflow”) is determined by integrating around the first loop, and shift volume weighted by the lung volume, which is derived from integrated airflow, (so-called volume-weighted-average shift volume) is determined by integrating around the second loop. sREFF can now be calculated as the quotient of volume-weighted-average airflow by volume-weighted-average shift volume. It therefore approximately indicates the volume-weighted-average airway resistance. An important limitation however, is that this volume-weighted average is derived not from true changes in thoracic gas volume, but rather from integrated airflow at the mouth.

sREFF provides improved signal-to-noise ratio over sRTOT. But on the other hand and more importantly, it is remote from primary WBP data. Details of the sRAW loop are hidden by the complex, multiple averaging that includes, at least, forming the ratio of two integrals of primary WBP data parameterized by values from a further preliminary integration of primary data. Also, despite its integrative character, it reflects more prominently resistance only in the larger central airways.

Other numerical parameters are known for characterizing sRAW loops. These include “instantaneous” values of airflow resistance provided by real-time, computer-assisted plethysmography. Another measure is sR0.5, which is the slope of the sRAW loop from 0.5 L/s inspiratory flow to either zero flow or to 0.5 L/s expiratory flow, which reflects the slope of the relatively linear portion of the sRAW loop. See, e.g., DuBois et al. 1956, A new method for measuring airway resistance in man using a body plethysmograph: values in normal subjects and in patients with respiratory disease J Clin. Invest. 35:327-335. Although sR0.5 standardizes the flow at which resistance is measured, this approach provides less inter-individual variability, because, both in normal subjects and in patients with airflow obstruction, resistance is dependent upon flow rate. Also, sR0.5 is primarily sensitive to the larger airways but much less sensitive to the peripheral airways.

Although these attempts to characterize sRAW loops can be used for assessment of normal patients, for comparison to normative data, for assessment of acute bronchial and therapeutic challenge, and the like these linear approximations provide only a limited capacity for the understanding of lung pathophysiology. Since all the afore-mentioned parameters manifest interpretative compromises in advanced obstructive lung disease, reliable characterization ultimately requires manual interpretation of the shape of actual sRAW loops. Additionally, all have their own particular calculational and numerical problems and peculiarities.

Airflow Perturbation Techniques

Respiratory airflow perturbation techniques (“RAP”) can be performed during a subject's normal, spontaneous breathing. These techniques determine mechanical properties of the airways and lung by measuring changes in respiratory airflow characteristics in response to repeated, small, external airflow perturbations. The respiratory airflow characteristics measured include mouth pressure, mouth airflow, and the like; the external airflow perturbations include changes in air pressure, air flow resistance, and the like. Exemplary RAP techniques include forced oscillation techniques (“FOT”) and techniques using airflow perturbation techniques (“AFP”).

FOT techniques apply oscillating external pressure signals to a subject's normal breathing and measure the oscillatory respiratory flows (“VRS”) arising from the oscillating external pressure. Several forms of FOT are known. For example, the external pressure signals can be either mono- or multi-frequency and can be applied either continuously or intermittently as pulses (impulse oscillation (“IOS”)). The FOT can be applied to pediatric, adult and geriatric populations for purposes of diagnostic clinical testing, monitoring of therapeutic regimens, and epidemiological evaluations. The FOT is also applicable to veterinary medicine.

In IOS, an aperiodic multi-frequency waveform (“pulses”) is used to provide data on lung mechanical properties over a continuous frequency range. Commonly, IOS pulses include frequencies from about 5 Hz to about 30 Hz and are repeated at rates of 3 Hz to about 5 Hz. Flows due to IOS pulses are separated from normal respiratory flows by modifying individual pulses with interpolated “baseline” straight line segments. Flows so determined that do not fulfill defined reliability criteria are rejected. IOS equipment, therefore, includes systems to apply pressure pulses with selected envelopes and sensitive respiratory flow measurement systems with the requisite bandwidth. IOS applied pressure (“PRS”) and resulting flow data (“VRS”) is processed by dividing the Fourier transform of PRS by the Fourier transform of VRS to determine the respiratory input impedance as a function of frequency. Respiratory impedance includes resistive and capacitive components.

AFP techniques apply periodically repeating perturbations to a subject's external respiratory airflows and measure changes in airflow and mouth pressure arising from these perturbations. A common AFP technique uses an airflow perturbation device (“APD”) to periodically alter the airflow resistance faced by a subject's respiratory airflows. For example, in an exemplary AFP technique, the subject breathes through a mouthpiece and the flow resistance through the mouthpiece is changed at a frequency of, e.g., 5 Hz to 15 Hz. Resulting changes in respiratory airflow rate and mouth pressure can be analyzed, as in IOS, by a ratio of Fourier transforms to determine the resistive and reactive components of the subject's airflow impedance at the frequency of the changing external airflow resistance. More commonly, flow and pressure changes are simply divided in the time domain to determine the subject's pulmonary airflow resistance. In detail, subject's internal pulmonary airflow resistance can be determined from the external flow and pressure perturbations because the periodically applied and known, external airflow resistance acts in series with the internal pulmonary resistance.

Lung pathology often manifests in abnormalities of the respiratory impedance spectrum due primarily to regional inhomogeneities in airway and lung mechanical properties. The resistive component of respiratory impedance (“RRS”) includes contributions primarily from the airways. When proximal (central) or distal (peripheral) airway obstruction occurs, resistance at 5 Hz (“RRS5”) is increased above normal values. The site of airway obstruction is inferred from the pattern of RRS increase: proximal airways obstruction elevates RRS evenly independent of frequency; distal airways obstruction elevates RRS primarily at lower frequencies (resistance at approximately 5 Hz, “RRS5”) with less elevation at higher frequencies. The reactive component of respiratory impedance (“XRS”) reflects inertia of the air column in the conducting airways and elastic (capacitative) properties of lung periphery. In both fibrosis and emphysema, low frequency capacitive reactance at 5 Hz (“XRS5”) is reduced: in fibrosis because of the stiffness of the lung; in emphysema because of partial peripheral airway obstruction.

Another, parameter conveniently determined by AFP is the resonant frequency (“FRES”) of the airway-lung system. FRES is often used as a marker to separate low-frequency from high-frequency XRS. In normal adults, FRES is usually 7-12 Hz; in healthy children, FRES is larger than in adults, increasing with decreasing age. In both obstructive and restrictive respiratory disease, impairments of the distal respiratory tract cause FRES to increase above normal. FRES has also been found useful to track within-subject trends over time during bronchial or therapeutic challenge.

Measuring accurate AFP data usually requires that a subject perform particular physical maneuvers. These maneuvers can be difficult for many subjects, and accordingly AFP data is often contaminated with noise and artifacts due to less than ideal subject behavior. In the art, such noise and artifacts have to be recognized by trained personnel who manually review AFP data.

As apparent from the background above, both WBP and AFP as currently practiced have certain problems. Data from WBP, the sRAW loops, is difficult to reliably and compactly interpret for clinical use. Data from AFP can be distorted by subtle aspects of patient behavior during testing.

### SUMMARY OF THE INVENTION

Objects of this invention are to provide improved methods for processing data from certain pulmonary function tests that overcome the problems encountered in their practice according to the prior art by providing novel systems and methods for the analyses of pulmonary function data.

More specifically, the invention provides improved methods for analysis of data obtained from WBP techniques and/or from RAP techniques (i.e., FOT, AFP devices, and the like). The improved computer-implemented methods provide improved characterizations of airway resistance determined by WBP. The improved computer-implemented methods also automatically recognize RAP data that has been distored by patient behaviors during testing, thereby obviating the need for manual review. This invention also provides computer systems that interface to devices that perform whole body plethysmography and/or the forced oscillation technique and automatically execute the methods of this invention. This invention also provides for distribution of software that causes computer systems to perform the methods of this invention.

The improved WBP methods of this invention depend directly on primary WBP measurement data; in fact, they require only a single integration of this data. In contrast, many prior art methods do not in fact depend on primary, scientifically-validated WBP measurement data; such methods require several, often complex, processing steps.

Turning to improved WBP methods, these methods characterize sRAW in new ways that are advantageous when compared to the prior art. In particular, the advantages quantify the effects of pathology of small, peripheral airway partial and complete obstruction. These methods characterize specific airway resistance loops by a numerical parameter reflecting loop area that is determined by integrating a weighting function within the loop. In a preferred embodiments, a constant weighting function is used so that the determined parameter depends only on the area within the loop. In alternative embodiments, a variable weighting function which depends on loop structure is used. Such a function can depend, for example, on the local orientation of the loop compared to overall orientation of the loop (e.g., local orientation compared to sRTOT) so that the resulting weighted integrative parameter estimates non-linear components. (Such a weighted parameter can also be determined by integrating around the edge of the loop.) Finally, the integrative numerical parameter can be normalized by constants determined from overall loop structure. One such normalization constant can be the range of shift volume (in ml.) spanned by the loop so that the normalized integrative parameter estimates loop hysteresis.

Turning to RAP, data produced by routine RAP testing, in particular by FOT and/or IOS and/or AFP, can contain artifacts and distortions. Recognizing such flawed data has typically required personnel trained to recognize the characteristics of common artifacts and distortions. The computer-implemented methods of this invention can automatically process AFP testing data and recognize the presence or absence of artifacts and distortions without manual analysis. Accordingly, routine RAP testing can be performed more efficiently both for the testing of single subjects and for the screening of many subjects.

Generally, RAP artifacts and distortions have recognizable patterns. This invention recognizes these patterns by separating them into their defining features, searching for these features in input data, and determining the presence or absence of artifacts and distortions on the basis of recognized features. The latter determination is rule-based in preferred embodiments. Other known pattern recognition techniques can be applied in alternate embodiments.

Preferred embodiments recognize artifacts and distortions that arise from subject behaviors, voluntary or involuntary, that lead to faulty transfer of pressure pulses from the AFP device to the subject's mouth, or to faulty transfer of pressure pulses from the subject's mouth to the subject's trachea. A common behavior of the first type is airflow leaks (“leaks”) that may occur around the mouthpiece, or with incompletely occluded nares. Common behaviors of the second type are tongue position artifact (“TPA”) in which the subject's tongue is unusually elevated, and dysfunctional vocal cord adduction during expiration (“VCAE”) in which the subject's vocal cords move in synchrony with respiration, in particular adducting in an exaggerated manner during expiration. Features of leak, TPA, and VCAE patterns and rules for recognizing these artifacts from found features are described herein. Other artifacts and/or distortions can be recognized by providing additional features and rules.

The methods and systems of this invention can also be configured for processing data from multiple WBP and/or RAP testing centers. These centers can test subjects in pharmaceutical studies, can perform population and epidemiological studies, can process data for multiple centers without local processing resources, and the like.

This invention also includes software products implementing the method of this invention. Hardware systems variously configured to perform the methods of this invention are also included.

Further aspects and details and alternate combinations of the elements of this invention will be apparent from the following detailed description and are also within the scope of the inventor's invention.

### BRIEF DESCRIPTION OF THE DRAWINGS

The present invention may be understood more fully by reference to the following detailed description of the preferred embodiment of the present invention, illustrative examples of specific embodiments of the invention and the appended figures in which:

FIG. 1A illustrates a computer system of the present invention linked to a whole body plethysmograph;

FIG. 1B illustrates a method of the present invention for processing a whole body plethysmographic data;

FIG. 2 illustrates exemplary airway resistance data;

FIG. 3 illustrates a computer system of the present invention linked to devices for performing AFP techniques;

FIG. 4 illustrates a method of the present invention for processing forced oscillation technique data;

FIG. 5 illustrates exemplary forced oscillation technique data;

FIG. 6 illustrates exemplary forced oscillation technique data;

FIG. 7 illustrates exemplary forced oscillation technique data;

FIG. 8 illustrates exemplary forced oscillation technique data;

FIG. 9 exemplary dysfunctional vocal cord adduction;

FIG. 10 illustrates exemplary dysfunctional vocal cord adduction;

FIG. 11 illustrates a system of the invention; and

FIGS. 12A-B illustrates further exemplary forced oscillation technique and/or airflow perturbation device data.

### DETAIL DESCRIPTION OF THE PREFERRED EMBODIMENTS

Preferred embodiment of the systems and methods directed to whole body plethysmography (“WBP”) and now described, and are followed by description of preferred embodiments of systems sand methods directed to respiratory airflow perturbation techniques (“RAP”), and finally of systems for implementing the methods of the present invention. The term RAP is used generically herein to refer to various specific techniques with perturb a subject's respiratory airflow. RAP techniques include those that more or less indirectly perturb respiratory airflow, for example, forced oscillation techniques (“FOT”) and impulse oscillation techniques (“IOS”) techniques, as well as those that more or less directly affect respiratory techniques, for example, airflow perturbation techniques (“AFP”) using airflow perturbation devices (“AFD”). Acronyms and generic groupings are used as described herein solely to facilitate and clarify description of the invention, and not with any limitation (e.g., arising from definitions known in the art).

Whole Body Plethysmographic Data Processing

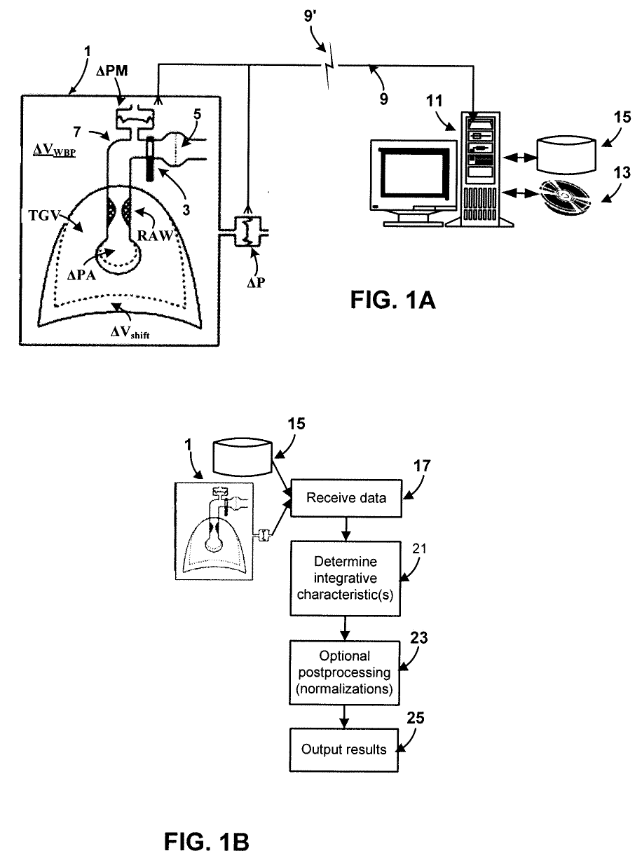

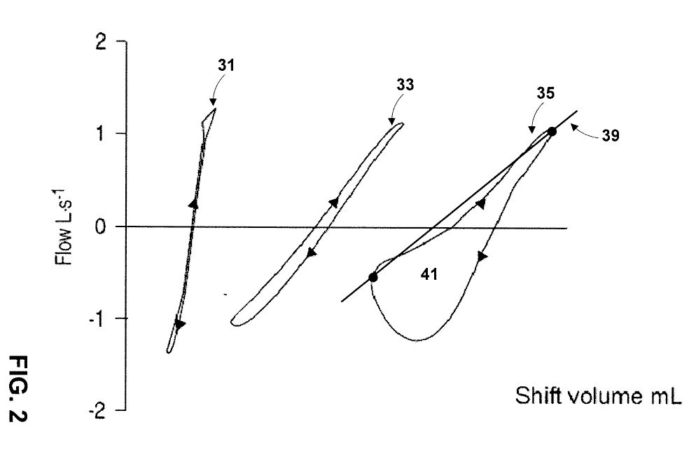

FIG. 1A illustrates constant-volume whole body plethysmograph having subject chamber 1 within which a subject being tested. Here, the subject is represented simply by a schematic pulmonary system, including a left and right lung, a single airway, and a single alveolus. The subject breathes through tube 7 that opens into the WBP chamber. Two (or more) pressure sensors measure the pressures indicated as ΔPM (pressure at subject's mouth) and ΔP (pressure in chamber 1). A pneumotachograph 5 measures airflow (by a pressure drop across a screen). Controllable shutter 3 occludes breathing tube 7 when activated. These sensors and controls are preferably linked directly 9 or indirectly 9′ to computer 11.

The principles and methods of finding airway resistance using a constant-volume WBP are now briefly described. As the subject breathes through tube 7 opening into the interior of the WBP chamber, inhaled and exhaled gas, the tidal volume Vₜ, moves back and forth between the WBP chamber outside the subject and the subject's lungs. Accordingly, subject respirationpre se does not change the total volume of gas within the WBP, that is the sum of the gas volume within the WBP chamber but outside the subject and the total gas volume within the subject's lungs (referred to herein as the “TGV”).

However, during inspiration, pressure in a portion of the TGV decreases, causing air to be sucked into the lungs from into the WBP chamber, while during expiration, pressure in this portion of the TGV increases, causing air to be forced out of the lungs and back into the WBP. These pressure changes during respiration cause a relatively small change in TGV known as the shift volume, ΔV_{shift}, which is illustrated as the portion of the lung within the outer solid boundary line (the TGV) but without the inner dashed boundary line. Now, ΔV_{shift}, being caused by pressure changes internal to the subject's lungs, causes a corresponding change in the gas volume, ΔV_{WBP}, and gas pressure, ΔP, inside the WBP chamber but outside the subject. ΔP is readily measured with the illustrated pressure sensor opening into chamber 1.

ΔV_{shift} is an important parameter because it is known to be directly correlated to changes in pressure in the terminal alveolar portions of the airways, ΔPA. ΔPA is in turn important because it is the pressure difference between alveolar pressure, PA, and mouth pressure, PM, that drives respiratory airflow, V′. ΔPM is also readily measured with the illustrated pressure sensor opening into breathing tube 7. The relation among ΔP, ΔV_{shift}, and ΔPA is readily calibrated by closing shutter 3, thereby occluding tube 7, and instructing the subject to make breathing efforts with his throat open normally, but against the closed shutter. Under such conditions of no airflow, ΔPM is directly related to ΔPA; ΔP is directly related to ΔV_{shift}; and putting together these coupled changes, ΔV_{shift} can be used to determine TGV from PA and ΔPA. The relationship between ΔPA and ΔV_{shift} is assumed to be the same during breathing with shutter open as it is during breathing efforts with the shutter closed.

Having calibrated the relation among ΔP, ΔV_{shift}, and ΔPA, airway resistance, RAW, is now readily measurable. RAW is the ratio of the total respiratory airflow, readily measured by pneumotachograph 7, divided by the pressure difference between the mouth, ΔPM, and the alveoli, ΔPA. It is noted that ΔPA is only derived from ΔV_{shift} after completion of the closed-shutter breathing efforts. These efforts are quite difficult for many subjects to perform. Accordingly the parameter sRAW is commonly used to provide an index of the subject's RAW multiplied by the subject's TGV to give specific airway resistance, sRAW, without doing the complex, coordinated respiratory effort maneuvers against the closed shutter, while trying to maintain the throat normally open. sRaw provides clinically useful information regarding the subject's airways, but has hitherto been interpreted by only a single estimate of the slope of the sRAW loop determined between one set of the afore-mentioned local points on the loop.

Turning now to the illustrated computer system, specific airway resistance data obtained from a WBP as just described, or otherwise obtained from a WBP or other instrumentation, is forwarded to computer 11 for processing. In certain embodiments, computer 11 is directly linked to a nearby WBP by a serial or parallel interface or a computer network 9. In certain other embodiments, airway resistance data is indirectly 9′ sent to computer 11. For example, data can be sent over the Internet in real time or in batches. Data can be stored on recordable media, for example disks or tapes or other types of computer-readable memories, and then physically transported to computer 11. Disk 13 can contain such data. Data can also be stored on database 15, which can be local or remote from computer 11, before and/or after processing by computer 11. Data can also be provided from one or more than one WBP and can also related to one or more that one subject.

With reference to FIG. 1B, after receiving 17 forwarded data, computer 11 finds and displays sRAW and/or RAW according to methods illustrated in FIG. 1B. Where the data includes measurements from WBP sensors, the computer determines ΔV_{shift} from ΔP, then ΔPA (if closed-shutter breathing efforts have been performed), and finally sRAW and/or RAW from the determined ΔPA, and measured ΔPM and respiratory air flow. These determinations can be made by the methods described above. Alternatively, ΔV_{shift}(and/or ΔPA) and sRAW and/or RAW may be determined previously and forwarded to computer 11.

Most commonly, specific airway resistance is graphically displayed as a function of ΔV_{shift} throughout one or more respiratory cycles, and the computer advantageously displays or prints such graphs on command. Because steady respiration is periodic, these graphs appear as closed loops of various shapes. FIG. 2 schematically illustrates several such loops from subjects having a variety of pulmonary pathologies. The graph plots respiratory airflow (V′) on the vertical axis and relative ΔV_{shift} on the horizontal axis. The arrows indicate progress of a respiratory cycle around the loop, where positive V′ occurs during inspiration and negative V′ occurs during expiration. Steeper loops or loop segments indicate lower airflow resistance, and conversely.

Loop 31 is from a subject with normal pulmonary function. A normal loop generally has a steeper slope indicating lower airway resistance, and has a narrow, to even substantially absent, width indicating that the behavior of the airways in inspiration and expiration are closely similar and that airflow resistance is nearly independent of lung volume and airflow rate. Loops 33 and 35 are from subjects with lung pathologies. Loop 35 arises from airways that behave significantly differently at different times in the respiratory cycle. Such differences in airway behavior are due to damage to smaller airways as occurs in chronic obstruction. Loop 33 has a decreased slope indicating increased airway resistance. This width, which is apparent but not large, indicates that the increased resistance is in the large airways because they tend to behave quite similarly during both inspiration and expiration.

In the prior art, various approximate measures have been used to characterize sRAW loops, such as loops 33 and 35. Loop 35 illustrates one such prior art measure, sRTOT, which characterizes loops by a single number, the slope of straight line 39 drawn between the extreme values ΔV_{shift}(indicated by the solid circles). sRTOT, a measure of an average airway resistance over an entire respiratory cycle, clearly fails to provide any information on differences in resistance between expiration and inspiration.

Accordingly, the present invention, in step 21, processes an sRAW loop to determine improved integrative characteristics that convey more complete information about the entire loop. In preferred embodiments, integrative characteristics include the area of the loop, which can be determined by standard numerical integration techniques. Loop area directly depends on differences in airway function occurring throughout the respiratory cycle. For loops similar to loop 35, loop area can be measured before and after a therapeutic intervention, and the differences found (if any) will be determined by changes (if any) in the mechanical behavior of the small peripheral airways. An additional characterization of loop behavior is, for example, by an integrative characteristic, such as an area, and can serve to distinguish among loops 31, 33 and 35. In alternative embodiments, the integrative characteristic can be the local slope integrated around the sRAW loop, or the (relative) standard deviation of the local slope from an average slope (i.e., as determined by integrating the local slope around the loop, or simply by sRTOT). It is clear from inspection of loop 35 that the local slope may include zero (at the bottom of the loop) or may be negative, indicating compression of thoracic gas behind closed airways. Such an integrative characteristic reflects the non-uniformity of the airway resistance throughout the respiratory cycle and the influence of closure of small peripheral airways. This characteristic can distinguish, e.g., between loops 31, 33, and 35.

Step 23 then determines one or more of the above integrative characteristics, as well as alternative or additional integrative characteristics that may be defined from time-to-time. Also, the integrative characteristics already determined in step 21 can be combined with other novel characteristics of this invention or with characteristics known in the prior art, i.e., sRTOT. This invention also includes other processing structures for determining and/or combining and/or normalizing characteristics

Finally, step 25 outputs the determined novel characteristics, for example, by direct display and/or printout and/or database storage, or similar.

Respiratory Airflow Perturbation Data Processing

Exemplary devices and the operation are now described for performing the forced oscillation technique (“FOT”) or its variant the impulse oscillation technique (“IOS”) and air flow perturbation using airflow perturbation devices (“APD”).

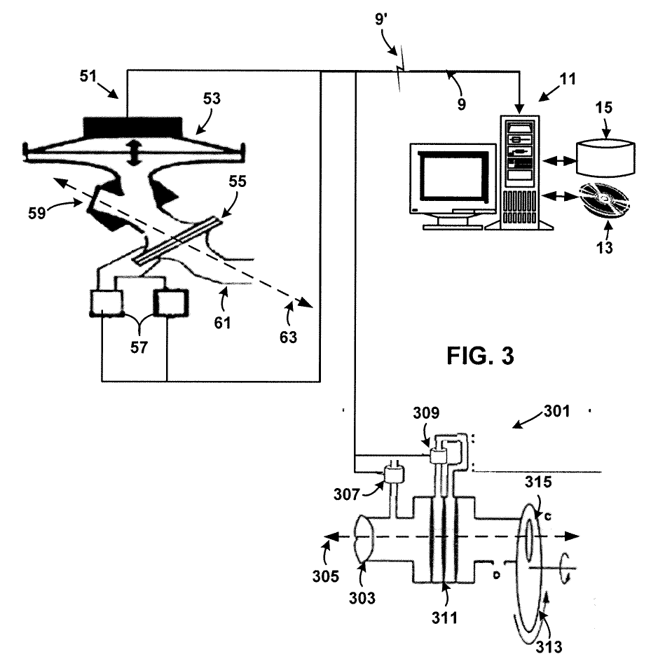

FIG. 3 illustrates key elements of impulse oscillation system (“IOS”) 51 and of airflow perturbation devices (“APD”) 301 with routine features being omitted. Considering first IOS system 51, a subject being tested breathes normally through tube 61, commonly with a microbial filter and mouthpiece interposed between the mouth and tube 61. Tube 61 opens to surrounding air though an acoustic resistor 59, and arrow 63 represents airflow generated by the subject's normal inspirations and expirations. Element 53 is similar to an audio loudspeaker and generates pressure waves (forced oscillations) that are superimposed on the subject's normal breathing. Generally, in FOT, the generated pressure waves are varied sequentially or randomly in frequency between limits of approximately 0 and 50 Hz. In IOS, the pressure waves of many frequencies are presented together as discrete pressure pulses. Pulse repetition rates vary most commonly between limits of approximately 3 and 5 Hz in present usable IOS systems, and the pulses are shaped to contain a superposition of frequencies within and/or including the above limits of approximately 0 and 50 Hz. The acoustic resistor is chosen to properly tune the acoustic response of tube 61 over the measured frequency range while allowing simultaneous respirations through with minimal impedance.

Total airflow, regular inspirations and expirations of respiration as well as flows due to pressure pulses generated by element 53, are measured by pneumotachograph 55. Airflow is determined from a pressure drop across the illustrated screen measured by pressure sensors 57. Pneumotachograph 55 preferably has a known, and preferably linear, frequency response over a range including 0-50 Hz. IOS 51 is preferably controlled by an electronic controller, which can optionally be computer system 11 that also performs the methods of this invention. The controller provides signals to element 53 (and other controllable elements) and receives signals from sensors 57 (and other sensors).

Raw IOS measurements, primarily a moment-by-moment record of airflow and air pressure in breathing tube 61, are processed to provide the frequency response of the subject's pulmonary system. First, the raw measurements are pre-processed to remove or ignore artifacts and to remove the effects of normal respiration from the airflow (and air pressure) measurements. After this pre-processing, IOS data reflect, to the extent possible, only the moment-by-moment pressure and airflow due to the superimposed pressure oscillations and/or pulses. This data is then Fourier transformed (preferably by a fast Fourier transform) into air pressure and air flow amplitudes and phases at frequencies throughout the measurement range, i.e., between approximately 0 and 50 Hz. The acoustic impedance of the pulmonary system at each frequency is defined to be the quotient of the Fourier transformed air pressure and Fourier transformed air flow at the measured frequencies. Acoustic impedance versus frequency data is then converted as known in the art into a resistive component (R, the “real” part of the impedance) and a reactive component (X, the “imaginary” part of the impedance).

Considering now APD system 301, a subject being tested breathes normally in and out 305 through breathing tube 303, commonly with a microbial filter and mouthpiece interposed between the mouth and breathing tube 303. At or near the end of breathing tube 303 that is away from the subject, a variable air flow resistor periodically changes the flow resistance faced by subject airflow 305. In the illustrated embodiment, the variable airflow resistor is disc 313 occluding the far end of the breathing tube and having regions 315 of greater or lesser airflow resistance, e.g., by being formed of screen with various mesh sizes. Disc 313 is spun at a rate of, e.g., 5 Hz to 15 Hz and thereby causes the airflow resistance though the breathing tube to vary at a similar rate.

Respiratory airflow and mouth pressure changes are measured as above. Mouth pressure is measured by pressure transducer 307 sensitive to the pressure difference between the interior of the breathing tube and the atmosphere. Airflow is determined from pressure transducer 309 sensitive to the pressure drop across screen 311. APD 301 also is preferably controlled by an electronic controller, either a local controller or a computer system such as system 11 that also performs the methods of this invention. The controller provides signals to controllable elements, such as to a motor spinning disc 313, and receives signals from pressure transducers 307, 309, and other sensors.

Raw APD measurements include primarily a moment-by-moment record of airflow and mouth pressure, and when graphed, appears as normal breathing on which are superimposed individually separated, coincident pairs of pressure and flow pulses arising from the airflow resistance perturbations. These raw measurements can be optionally pre-processed to remove artifacts. Commonly, the raw (or pre-processed) data is then analyzed by dividing perturbed respiratory pressure and flow at the times of the coincident pairs of pressure and flow pulses and also by dividing normal respiratory pressure and flow at the times between the coincident pairs of pressure and flow pulses. From these pairs of resistance values, a time domain record of the subjects pulmonary resistance at the perturbation frequency can be determined. Additionally, a reactance estimate can be determined from the phase difference (if any) between the perturbed pressure and flow. The phase difference can in turn be determined by closely comparing the difference in start times of the coincident pairs of pressure and flow pulses. Alternatively, the raw (or pre-processed) data can be analyzed in a manner similar IOS data analysis by Fourier transformation.

The primary output of an AFP measurement session (e.g., by an FOT device, or an IOS device, or an APD) is the subject's respiratory resistance and/or impedance (including reactive components) versus frequency over the measurement range. In the case of IOS measurements, the frequency range can continuously extend from a few Hz to about 50 Hz or so. In the case of APD measurements, the frequency range includes primarily those discrete frequencies at which airflow was perturbed. Additional parameters can also be extracted from the primary signals. For example, respiratory volumes can be determined from the time integral of the measured airflow.

It will be appreciated from the above that certain subject behaviors, either voluntary or involuntary, can result in AFP data (e.g., FOT or IOS or AFP data) with distortions and/or artifacts. Among such behaviors are those that lead to faulty transfer of pressure pulses from the IOS device to the subject's mouth, and those that lead to faulty transfer of pressure pulses from the subject's mouth to the subject's airways. Transfer of AFP flow perturbations to the subject's airways can be faulty for similar reasons. A common behavior of the first type is inadequate hold of mouthpiece attached to tube 61 leading to airflow leaks (“leaks”) around the mouthpiece. Common behaviors of the second type are the tongue position artifact (“TPA”) in which the subject's tongue is unusually elevated, and also, dysfunctional vocal cord adduction (“VCAE”) in which the subject's vocal cords move in synchrony with respiration, in particular adducting in an exaggerated manner during expiration, with some “hangover” into subsequent inspiration. Both these behaviors of the second type impede flow into and out of the airways.

In more detail, airflow leak during IOS measurements (and also during AFP measurements) leads to non-uniformities in the air pressure and/or airflow records that occur in synchrony and at the frequency of the applied pressure pulses. Leaks can occur in any subject, and can be corrected by additional subject instruction. TPA results when airflow is partially impeded due to incorrect placement of the patient's tongue. See, e.g., Goldman et al., 2005, “Clinical Applications of Forced Oscillation to Assess Peripheral Airway Function”, Respiratory Physiology and Neurobiology, v 148, p 179-94. TPA causes anomalously elevated R (resistance) where the anomalous increase in resistance is substantially the same at least over approximately the frequency range of 10-25 Hz. Such anomalous elevations in R are determined by comparisons with other respiratory resistance measurements of the same subject.

In VCAE the vocal cords adduct (approach closer to each other or partially close) during expiration in an exaggerated manner compared to normal, and thereby partially impede airflow when adducted. VCAE can therefore be recognized by the temporal pattern demonstrating a changing R during the respiratory cycle, in particular R values that increase late in expiration, and remain elevated until after the beginning of the following inspiration. VCAE may occur during normal breathing in subjects who are, e.g., middle-aged or older, who have asthma, and/or who have a chronic obstructive pulmonary disease (“COPD”), and the like. VCAE may also occur in conditions that irritate the vocal cords, including unusually dry upper airway lining tissue, allergic rhinitis, reflux of gastric acid, and the like. In some of these subjects, VCAE may cause no specific symptoms during normal breathing. In other subjects, VCAE may be associated with difficulty during inspiration, shortness of breath, inspiratory stridor, and the like, where it is known as vocal cord dysfunction (“VCD”).

Computer systems and computer-implemented methods of this invention automatically recognize the presence (or likely presence) of these and other artifacts and distortions in primary FOT and/or IOS data and/or AFP data. Thereby, such data can be flagged for later manual attention and/or excluded from further analysis. These methods are now generally described; following the general description, aspects specific to specific artifacts and distortions are described.

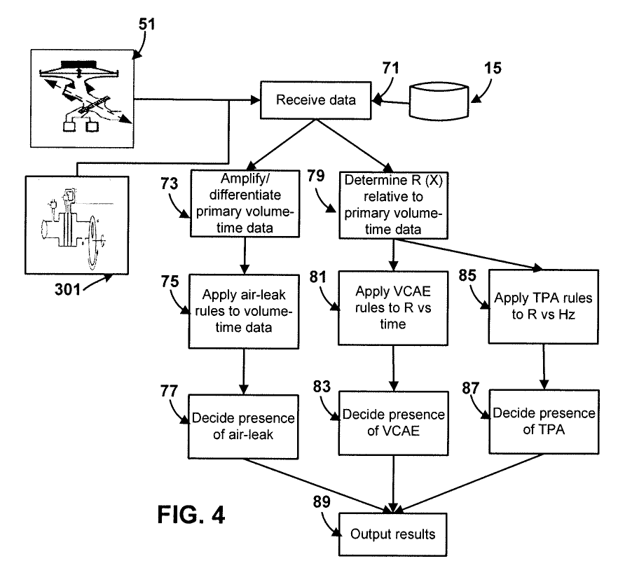

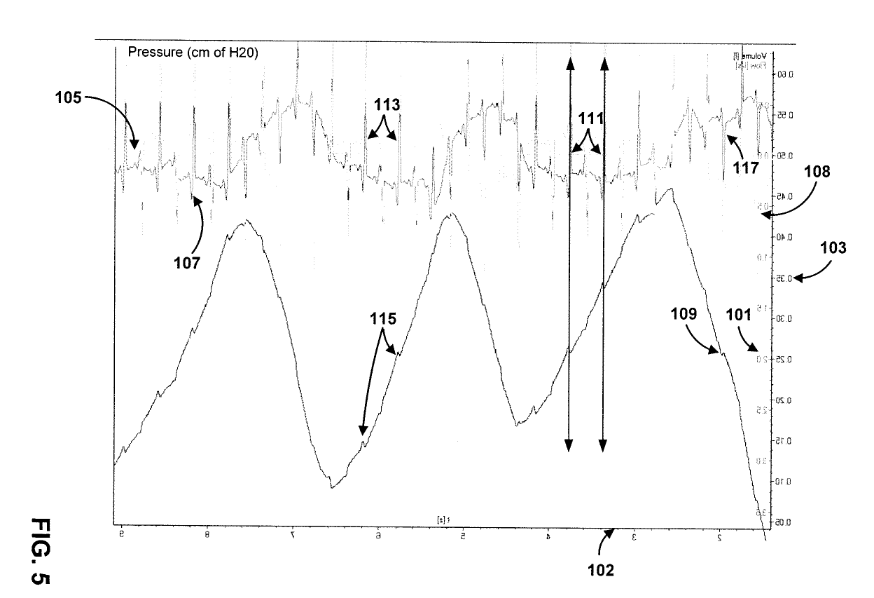

The computer-implemented methods can be performed on standard PC-type or workstation-type (or other type) computer system, for example, computer system 11, which can be part of an AFP measurement device or local to or remote from an AFP measurement device. FOT and/or IOS data and/or APD can be transferred (71 in FIG. 4) directly 9 or indirectly 9′ to system 11. In certain embodiments, computer 11 is directly linked to nearby IOS 51 or to nearby APD 301 by a serial or parallel interface or a network 9, so that FOT and/or IOS data and/or APD can be processed in real time if desired. In certain other embodiments, data is indirectly 9′ sent to computer 11, i.e. over the Internet in real time or in batches, or stored on computer-readable media, for example disks, tapes, memories and the like. Data can be provided from one or more than one IOS device and can related to one or more than one subject.

Most artifacts and/or distortions can be recognized because they lead to unique patterns in AFP data that are not present in normal data. In a preferred embodiments, artifact patterns are described in this invention by a set of pre-determined signal features; and the artifact is then recognized by the presence (or absence) in the data of a threshold number of features from the pre-determined set of features. For example, a particular pattern may be recognized by the presence of one feature and the absence of a second feature, or by the presence of half the features of the set, or by the presence of all the features of the set. In another preferred embodiment which is described below, pre-determined rules can be applied to the features found in received data, and the presence or absence of a particular artifact and distortion then recognized according to the rules. This invention is not to be limited to these preferred pattern recognition technique, but can also be implemented by other signal and/or pattern recognition techniques, i.e., neural networks.

With reference to FIG. 4, the described rule-based embodiments methods of this invention receive data 71 (from IOS 51, APD 301, or database 15), and then test (e.g., 73 and 79) the received data for the presence or absence features of interest that define the artifacts and distortions of interest. These tests use known signal processing techniques and/or the previously described techniques for processing IOS data. Next, the rules defining the various artifacts and/or distortions of interest are applied (e.g., 75, 81, and 85); the presence and/or absence of the artifacts is determined (e.g., 77, 83, and 87), and the results are output 89. While FIG. 4 illustrates the methods for the recognition of airflow leaks, TPA, and VCAE, the invention is not to be limited to recognition of these three artifacts. Rather, other sets of features and rules can be developed for other recognizable artifacts and/or distortions in IOS signals.

More specifically, data can be received 71 directly from measurement devices 51 or 301 and/or indirectly by being stored on recordable media or providing by communications. Step 73 processes and tests for features pertinent to airflow leaks, namely non-uniformities in airflow and/or air pressure that indicate the present of leaks. This step amplifies in amplitude and/or differentiates in time the primary IOS pressure and flow signals. Step 79 determines R and/or X values relative to received data as described above, since both TPA and VCAE are recognized by artifacts and/or distortions in R and/or X values.

Specifically, for airflow leaks, step 75 applies airflow leak rules to the amplified/differentiated data, and step 77 outputs the presence of an airflow leak, or the likelihood that an airflow leak is present, as determined by the rules. The key rule indicating an air-flow leak is the presence of non-uniformities in the temporal pattern of respiratory volume (found by integrating airflow, V′) that occur in synchrony with applied pressure pulses (or other airflow perturbations). If, for example, pressure pulses are applied at a rate of 5 Hz, non-uniformities occur at intervals that are multiples of 200 millisecond (msec.), for example, every 200 msec. or every 400 msec., and so forth. The duration of the non-uniformities is comparable to the duration of the pressure pulses, which is 10 msec. to a few 10's of msec. (To cover a bandwidth of approximately 0-50 Hz, the duration of a pressure pulse must be approximately 20 msec. (1/50)). These parameters can be immediately adjusted for different pulse rates and bandwidths. Typical respiratory volume non-uniformities include rapid, small amplitude volume fluctuations superimposed on the respiratory volume trace. These can be detected by comparing an actual volume trace with a volume trace which has been smoothed by interpolation (e.g., by linear interpolation, by spline interpolation, and the like) over times of 1 to 3 pressure pulse intervals. The key rule is modified to indicate more severe or even unacceptable airflow leaks when the volume trace has sawtooth marks in alternating directions towards increased and then towards decreased volume, and so forth.

A further preferred air-flow leak rule is abrupt changes in integrated airflow, either a reversal in integrated airflow or an abrupt increase in integrated airflow, that occur approximately synchronously with respiratory non-uniformities. These airflow features are usually readily observed as deviations in the integrated airflow trace having large amplitudes and durations approximately equal to the durations of the pressure pulses. Characteristically, the integrated airflow deviations have a duration of approximately 10 msec. to a few 10's of msec., occur every 200 msec., or 400 msec., or similar (for 50 Hz pulses and a 5 Hz rate), and have an amplitude large compared to noise and similar artifacts in the integrated airflow trace. A reversal of direction produces a spike directed oppositely to the current airflow direction, while an abrupt increase produces a spike directed in the current airflow direction. These features can also be readily recognized in the time derivative of the respiratory volume by finding discontinuities that include abrupt changes in algebraic sign, or abrupt transient increases in magnitude in close temporal association with the pressure pulses.

It is further preferable that an airflow reversal non-uniformity have a resultant volume change in the reversed direction of approximately greater than 10 ml in adults and approximately greater than 5 ml in children. Conversely, a slowing of flow without change in direction is not considered to be air flow leak. It is further preferred that an abrupt-increase-in-flow non-uniformities alternate with reversal-of-flow non-uniformities; each therefore occurring at intervals of 400 msec. (in the case of pulse at a 5 Hz rate), and occur after inspiratory (positive) airflow has risen above a threshold level but before expiratory (negative) airflow falls below the same threshold level.

According to a further rule, significant transient declines in computed respiratory resistance indicate airflow leaks whether or not these leaks are indicated by the other above-described rules. However, transient declines in computed respiratory resistance are optional. The absence of a computed respiratory resistance decline does not indicate that airflow leaks are absent if they are otherwise indicated by the other rules above-described.

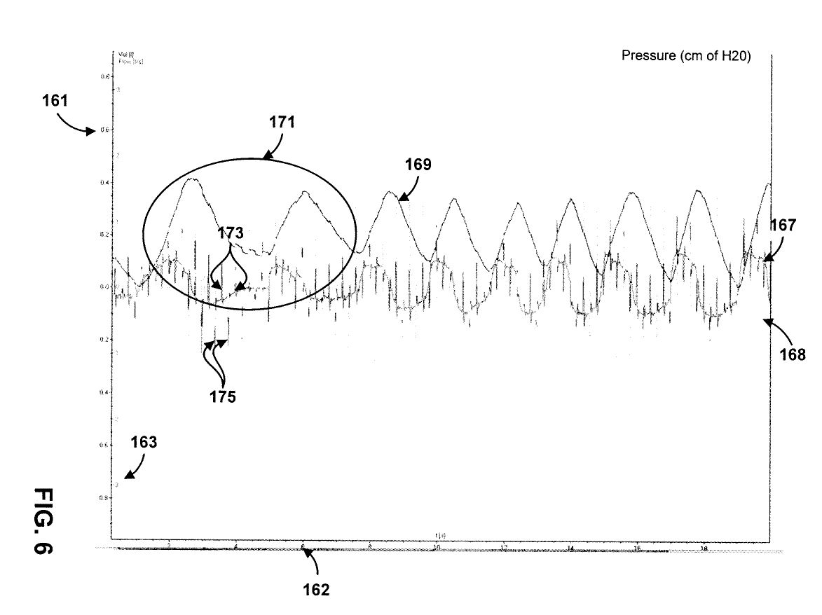

FIGS. 5 and 6 illustrates the patterns of non-uniformities arising in connection with air leaks described above. In particular, these figures illustrate non-uniformities in the temporal pattern of respiratory volume associated with pressure pulses and with patterns of alternating flow reversals and abrupt increases in flow. Generally with respect to FIG. 5, horizontal axis 102 indicates values of time in seconds; vertical axis at 101 indicates values of respiratory flow in liters/sec; vertical axis at 103 indicates respiratory volume, that is the integral of respiratory airflow, in liters; and dashed line 105 indicates zero airflow, and pressure. Further, trace 107 indicates respiratory airflow; trace 108 indicates pressure measured in the IOS device; and trace 109 indicates respiratory volume. And generally with respect to FIG. 6, horizontal axis 162 indicates values of time in seconds; vertical axis at 161 indicates values of respiratory volume, that is the integral of respiratory airflow, in liters; vertical axis at 163 indicates values of respiratory airflow, in liters/second; the dashed horizontal line indicates zero airflow, volume, and pressure; trace 167 indicates respiratory airflow; trace 168 indicates pressure measured in the IOS device; and trace 169 indicates respiratory volume.

Turning to FIG. 5 in more detail, respiratory volume trace 109 has a pattern of temporal non-uniformities characteristic of an airflow leak These non-uniformities, as required, occur in association with pulses in pressure (or flow perturbation) trace 108. Events 111, as highlighted by the adjacent vertical arrows, include non-uniformities in volume trace 109 at the time of pulses in pressure (or flow perturbation) trace 108. The volume non-uniformities further occur in association with reversals of flow. Here, positive going spikes are superimposed on a negative baseline airflow. Also, pressure and airflow non-uniformities 113 occur in temporal association with volume irregularities 115. The airflow non-uniformities are again a flow reversal with positive going spikes superimposed on a negative baseline airflow. Event 117 is similar to events 111 and 113 except that the flow reversal here is a negative going spike superimposed on a positive baseline airflow.

Turning to FIG. 6 in more detail, a more severe airflow leak occurs as indicated by oval 171. During this period non-uniformities in volume trace 169 include sawtooth marks having alternating directions, first towards increased and then towards decreased volume and so forth. As necessary, these volume non-uniformities occur in association with pressure pulses in pressure (or flow perturbation) trace 168. Examination of airflow traces 167 reveals increases in flow non-uniformities 175 where negative going airflow spikes are superimposed on a negative baseline airflow. As required, these increases in flow non-uniformities alternate with reversals of flow non-uniformities 173. At 173, positive going airflow spikes are superimposed on a negative baseline airflow.

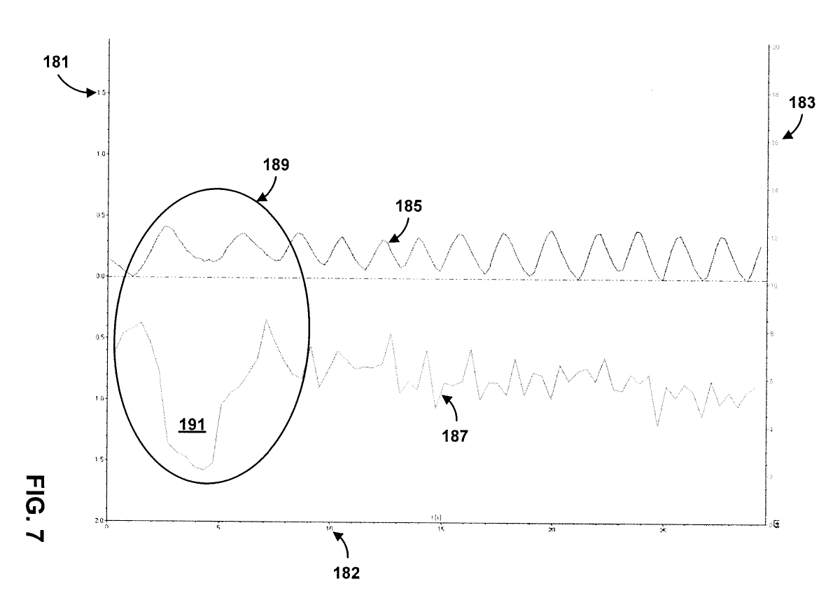

Finally, FIG. 7 illustrates a transient decline in computed respiratory resistance such as occurs occasionally in synchrony with and indicating airflow leaks, here applied to the same incoming data as illustrated in FIG. 6. The circle 189 includes the volume trace (shown in greater amplification in FIG. 6) and the resultant substantial transient decrease in calculated R, 191. In this figure, horizontal axis 182 indicates the values of time; vertical axis at 181 indicates respiratory volume, that is, integrated airflow, with the dashed line at zero volume change occurring at an approximate resistance value of 10.2 cm H20/L/s; vertical axis at 183 indicates respiratory resistance; trace 185 indicates respiratory volume (that is integrated airflow); and trace 187 indicates computed respiratory resistance. Oval 189 includes a significant airflow leak that is indicated by large drop 191 in respiratory resistance. Small volume non-uniformities can be seen through part, but not all, of this airflow leak.

Turning to the TPA, step 85 applies TPA rules to the respiratory resistance versus frequency data determined after further processing of data calculated in step 79. Then, step 87 outputs the presence of an airflow leak, or the likelihood that TPA is present. The presence or absence of TPA in a FOT or IOS measurement session is assessed by first determining respiratory resistance (R) between approximately 10 and 25 Hz. When the determined R values in the present measurement session are increased between approximately 10 and 25 Hz by a substantially constant amount independently of oscillation frequency compared to the R values from one or more previous measurement sessions, provided that no airflow leak is present in the previous measurement sessions, TPA is indicated to be present. R values in one session are increased by a substantially constant amount when compared with R values from another session, if the relative difference between R values at each frequency vary by at least approximately 10-20%, or another variation significantly greater than expected measurement variability. In alternative embodiments, R values can be assessed at a few discrete frequencies between approximately 10 and 25 Hz, for example, at 10, 15, 20, and 25 Hz.

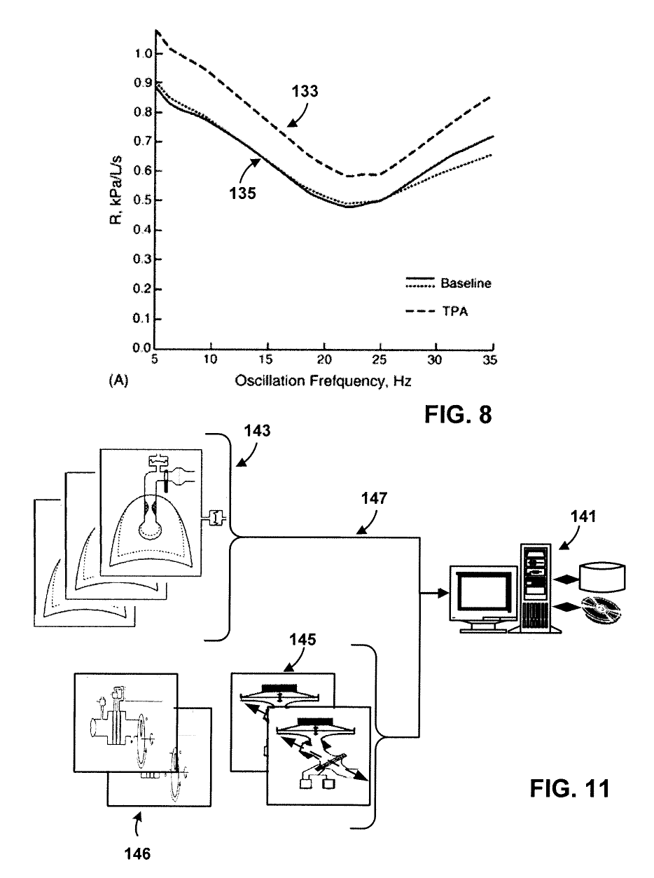

FIG. 8 illustrates a pattern of changes in the resistance (vertical axis) versus frequency (horizontal axis) relations associated with TPA. Traces 135 represent baseline resistance measurements (without airflow leak) where TPA was not present. Trace 133 represents a measurement in the presence of TPA. Trace 133 is substantially uniformly increased with respect to traces 135 over the preferred frequency range of 5 Hz to 35 Hz. The increase is approximately 0.1 kPa/L/s out of 0.5-1.0 kPa/L/s, or from 10% to 20%.

Turning to VCAE determination, step 79 determines respiratory volume versus time by integrating the airflow data, and also respiratory resistance versus time by the above-described methods. Step 81 compares these two curves and applies the VCAE rules, and step 83 outputs the presence of a VCAE artifact, or the likelihood that a VCAE artifact is present, according to the rules output. A characteristic pattern indicative of the presence of VCAE are changes of R (at one or more selected frequencies) occurring in synchrony with inspirations and expirations. In particular, R should increase during the latter part of expiration. In other words, a curve of R (at a selected frequency) versus time will change direction during expiration from decreasing with time to increasing with time, or from a decreasing rate of increase to an increasing rate of increase, or will show a decreasing rate of decrease in time, in the latter part of expiration.

Preferred features of the VCAE pattern include that the increase of R (at one or more selected frequencies) begins (or increases) after approximately midway through expiration, i.e., after the expired volume reaches approximately 50% of total expired volume. A further preferred feature is that R (at one or more selected frequencies) does not return to a subsequent minimum until after the beginning of the subsequent inspiration. A further preferred feature is that the increase in R (at one or more selected frequencies) relative to the immediately preceding nadir or inflection point in R be least 25% greater, or 100% or greater, or 500% or greater, or up to 1000-2000% greater. A further preferred feature is that the increase in R occur at frequencies greater than approximately 5 Hz, or greater than approximately 10 Hz, or greater than approximately 15 Hz, or up to approximately 25 Hz. In fact, since the determination of R using IOS manifests less random noise at higher frequencies, R can be preferably sampled and displayed versus time at frequencies greater than approximately 10 Hz or greater than approximately 15 Hz. Sampling at lower frequencies less than approximately 8 Hz is not preferred. A further confirmatory rule is that low-frequency reactance (X) between approximately 3-10 Hz may or may not increase in magnitude if concurrent relative changes in R are approximately 25 to approximately 50%. However, relative changes in R of 100% or more are commonly accompanied by simultaneous relative increases in low frequency X that can be from 50 to 1000% or more. A further confirmation rule is that when low-frequency reactance (X) increases in magnitude abruptly during the first 50% of expired volume followed by an abrupt decrease in magnitude during the latter 50% of expired volume, a concurrent increase in R in association with the decrease in magnitude of X is taken to indicate the presence of VCAE.

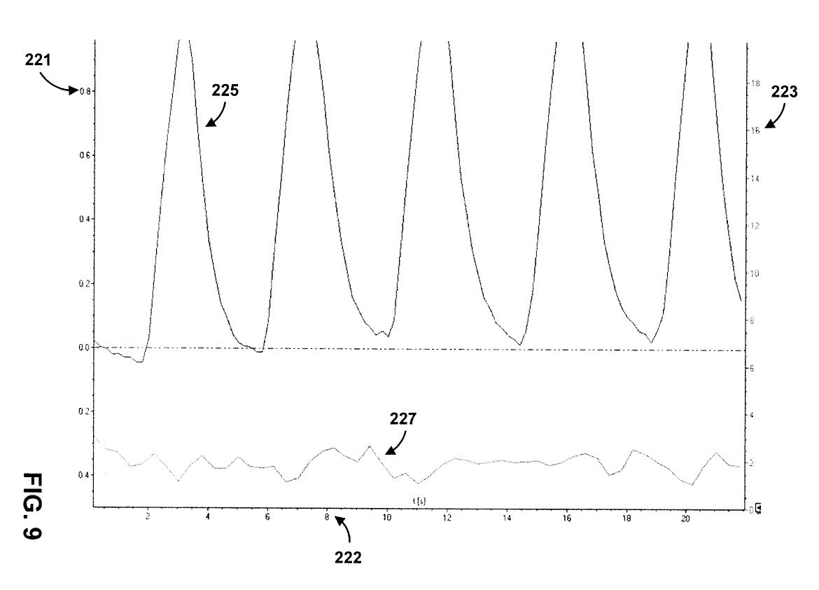

FIG. 9 illustrates a normal pattern of calculated respiratory resistance, R, and respiratory volume versus time. Here, horizontal axis at 222 indicates time values; vertical axis at 221 indicates values of respiratory volume with the dashed line at zero volume change; vertical axis at 223 indicates values of R; trace 225 indicates respiratory volume; and trace 227 indicates respiratory resistance, R. Here, R shows normal variable, nonsystematic, small changes during inspiration and expiration. There is no correlation between changes in R and respiratory volume.

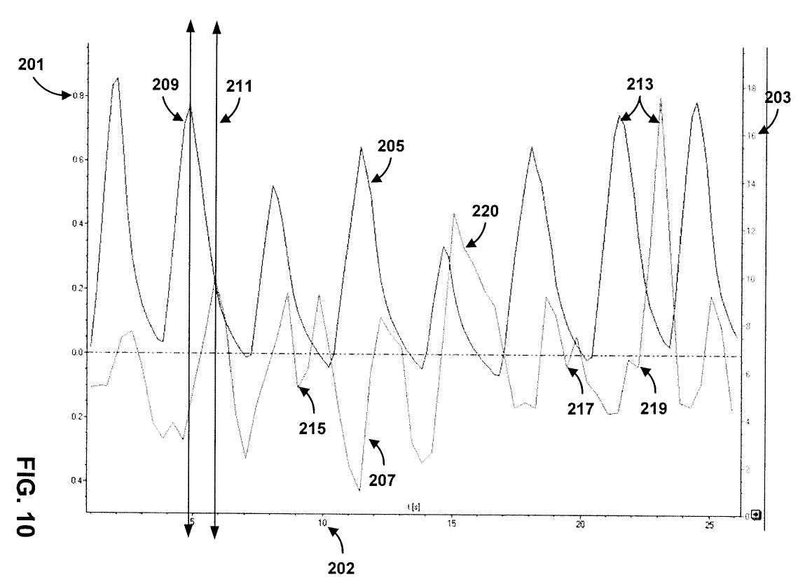

FIG. 10 illustrates the VCAE abnormality. Here, horizontal axis at 202 indicates time values; vertical axis at 201 indicates values of respiratory volume with the dashed line at zero volume change; vertical axis at 203 indicates values of R; trace 205 indicates respiratory volume; and trace 207 indicates respiratory resistance, R. Here, R shows systematic large variations during inspiration and expiration, which are strongly correlated with respiratory volume. Approximately, variations in R can be appreciated to lag variations in respiratory volume by from approximately one quarter to one third of a cycle up to approximately eight tenths of a cycle. That is R begins to increase, or its increase increases further, during the ending of expiration, that is usually at least after approximately midway through expiration. Also in most cases in this figure, R does not return to a subsequent minimum until after the beginning of the subsequent inspiration.

Specifically, the double arrows and 209 and 211 highlight that here the maximum of R lags the maximum of volume by approximately one half of a respiratory cycle (indicated in the volume trace), but no evidence exists at this point for VCAE. On the other hand, at 213, the maximum of R lags the maximum of volume by approximately eight tenths of a respiratory cycle. Furthermore, this maximum of R occurs subsequent to a much smaller local maximum early in this exhalation, and starting at 219. Respiratory resistance, R, at 215 (where the time is 9 sec) and at 217 (where the time is 19.5 sec), and at 219 (where the time is 22.5 sec) show examples of changes in R from decreasing with time to increasing with time during the latter part of expiration. And respiratory resistance, R, at 220 (where the time is 15.5 sec) shows an example of a decreasing rate of decrease in time.

Moreover, VCAE can be identified with these described techniques even in the presence of other pulmonary abnormalities. One such abnormality is known as expiratory flow limitation (“EFL”). See, e.g., US patent publication no. 2005/0178385 published Aug. 18, 1985. Briefly, properties of the normal lung and bronchi are such that expiratory flows are limited. In normal subjects, the expiratory flow limits are sufficiently high that reserve expiratory flow capacity is available for use during periods of exercise and the like. However, in subjects with lung pathologies, for example, COPD, expiratory flows can become so limited that even normal, resting respiration is at or near the expiratory flow limit. Such subjects then have clinical EFL, at rest, and it can be important for their treatment and management that resting EFL be identified and measured.

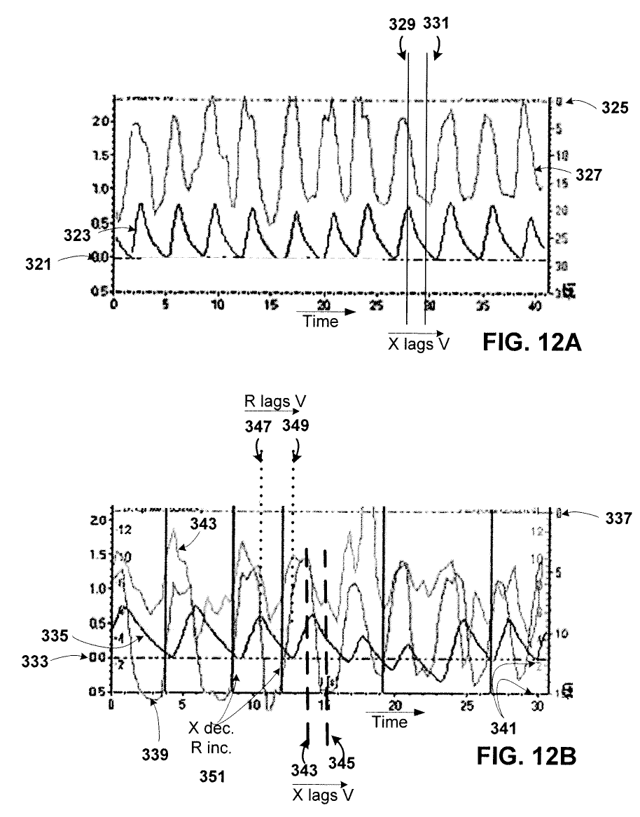

One sign of EFL is based on measuring the time course of a subject's pulmonary reactance. Subjects with pulmonary pathology often have lung reactance that periodically varies during the respiratory cycle, and EFL is then indicated if the phase of the varying reactance (X, measured by, e.g., an IOS technique) lags the phase of the tidal volume (V, measured by, e.g., integrating airflow). FIG. 12A illustrates a subject with EFL having a lagging reactance (increasing time is along the horizontal axis). In this figure, the right hand vertical axis measures reactance with zero line of reactance 325 (increasing downward), and the left hand vertical axis measures tidal volume with (increasing upward) zero line 321. The volume trace is 323, and the reactance trace is 327. Examination of this figure reveals that the maxima of the reactance trace (reactance increasing downward) lag the maxima of the tidal volume trace (tidal volume increasing upward). For example, a maximum inhalation occurs at 329 while the corresponding maximum reactance occurs subsequently at 331. Since “X lags V”, as lung volume decreases reactance increases.

VCAE can be recognized in a subject with resting EFL as before if airway resistance (R, measured by, e.g., an IOS technique or an APD device) is seen to lag tidal volume. FIG. 12B illustrates a subject with EFL in which VCAE can also be recognized (increasing time is again along the horizontal axis). In this figure, the volume scale (increasing upward) is again along the left hand vertical axis with zero volume line 333. The right hand vertical axis has outer ticks measuring the reactance (increasing downward) with zero line 337, and has inner ticks measuring the airway resistance (increasing upward) with zero line 341. Trace 335 presents the time course of tidal volume; trace 339 presents the time course of reactance; and trace 343 presents the time course of airway resistance. Close examination of this figure again reveals the presence of EFL because reactance is seen to lag tidal volume (i.e., “X lags V”). For example, maximum 345 of the reactance trace lags maximum 343 of the tidal volume trace. Further, the presence of VCAE is also apparent because the maxima of the airway resistance trace also lag the maxima of the volume trace (i.e., “R lags V”). For example, maximum 349 of the resistance trace lags maximum 347 of the tidal volume trace. Also, it is often the case, as here, that maxima of the resistance trace lag maxima of the reactance trace. In such cases, the reactance is deceasing when the resistance is increasing. This pattern of reactance-resistance changes is visible here at, e.g., the vertical black lines such as lines 351.

If necessary, automatic determinations by methods of this invention can be reviewed and/or corrected by manual inspection.

Clinical and Pharmaceutical Screening

The automatic, computer-implemented methods and systems of this invention are also advantageous for clinical and pharmaceutical screening applications. Clinical screening can be used for assembling typical test values from various types of subjects, for healthy monitoring and maintenance, for epidemiological studies, for research, and the like. Pharmaceutical screening can be used for determining pulmonary effects of drugs directed at pulmonary functions, for monitoring side effects of drugs directed at other organs, and the like. Additionally, these methods and systems can provide data analysis for multiple remote locations that perform WBP and/or RAP (e.g. IOS or AFP) testing but that do not have necessary processing capabilities.

FIG. 11 illustrates a system adapted to screening applications. Here, computer system 141 is directly or indirectly linked to locations performing pulmonary function testing. One or more locations 143 perform WBP testing; one or more locations 145 perform AFP (e.g., FOT or IOS or AFP) testing; and other locations can perform both WBP and RAP (i.e., FOT or IOS, or AFP) testing and/or testing according to methods. Methods of this invention executed on computer system 141 then process data sets received for subjects tested and automatically indicate where artifacts or distortions are likely to be found in the received data. Data with artifacts and distortions can then be excluded from further analysis. Combinations of raw and processed data can be stored in the associated illustrated local or remote databases.

This methods of this invention can be performed on software or firmware programmable systems. In the case of software programmable systems, methods are coded in standard computer languages, such as C, C++, or in high level application languages, such as Matlab and associated toolboxes (Math Works, Natick, Mass.). Code is then translated or compiled into executable computer instructions for controlling a microprocessor or similar.

The preferred embodiments of the invention described above do not limit the scope of the invention, since these embodiments are illustrations of several preferred aspects of the invention. Any equivalent embodiments are intended to be within the scope of this invention. Indeed, various modifications of the invention in addition to those shown and described herein, such as alternate useful combinations of the elements described, will become apparent to those skilled in the art from the subsequent description. Such modifications are also intended to fall within the scope of the appended claims. In the following (and in the application as a whole), headings and legends are used for clarity and convenience only. Further, the term “or” is used herein in the inclusive sense; that is “A or B” is to be understood as meaning one of “A”, “B”, and “A and B”.

A number of references are cited herein, the entire disclosures of which are incorporated herein, in their entirety, by reference for all purposes. Further, none of these references, regardless of how characterized above, is admitted as prior to the invention of the subject matter claimed herein.

What is claimed is:

1. A computer-implemented method for processing data from pulmonary measurements comprising:
receiving specific airway resistance data throughout one or more cycles of respiration;
determining an integrative measure characterizing the received specific airway resistance, the integrative measure depending on values of specific airway resistance throughout one or more respiratory cycles;
normalizing the integrative measure; and
outputting the normalized integrative measure.

2. The computer-implemented method of claim 1 wherein the integrative characteristic is the area enclosed by a graph of airflow at the mouth versus an indicia of alveolar pressure

3. The computer-implemented method of claim 2 wherein the indicia comprises shift volume.

4. The computer-implemented method of claim 2 wherein the indicia comprises alveolar pressure.

5. The computer-implemented method of claim 1 wherein normalizing comprises dividing by the total lung volume.

6. The computer-implemented method of claim 1 wherein normalizing comprises dividing by one or more of average the airway resistance, the average specific airway resistance, the total specific airway resistance (sRTOT), and the specific airway resistance 0.5(sRAW0.5).

7. A computer-implemented method of processing data from a whole body plethysmographic (WBP) device having sensors for pressure and airflow, the method comprising:
receiving data from WBP sensors characterizing at least respiratory pressure and respiratory airflow throughout one or more cycles of respiration;
determining airway resistance as a quotient of subject respiratory airflow and an indicia of subject alveolar pressure, which is also determined from the received data;
determining an integrative measure characterizing the determined specific airway resistance, the integrative measure depending on values of airway resistance throughout one or more respiratory cycles;
normalizing the integrative measure; and
outputting the normalized integrative measure.

8. A computer system comprising:
a processor;
a memory operatively coupled to the processor; and
a communications interface linked directly or indirectly to at least one WBP device,
wherein the memory comprises stored instructions for causing the processor to perform the methods of claim 7.

9. The computer system of claim 8 wherein the direct or indirect link between the communications interface and the WBP device comprises a network link.

10. The computer system of claim 8 wherein the direct or indirect link between the communications interface and the WBP device comprises a physically-transferable, computer-readable medium.

11. The computer system of claim 8 further comprising a WBP device linked to the communications interface.

12. A computer-implemented method for processing data from forced oscillation technique (FOT) measurements, the FOT technique superimposing periodic, short pressure pulses on a subject's respiratory airflow, the method comprising:
receiving pressure and flow data characterizing the pressure pulses applied to a measured subject throughout a measurement period;
determining from the received data the presence or absence one or more features that are indicative of the presence or absence of artifact or distortion in received data;
deciding that an artifact or distortion is present, or is likely to be present, in the received data in dependence on one or more of the determined features; and
outputting whether or not artifact or distortion is present or absent in the received data.

13. The computer-implemented method of claim 12 wherein determining features further comprises determining respiratory volume versus time data from the received data.

14. The computer-implemented method of claim 13 wherein deciding that an airflow leak artifact or distortion is present further comprises searching for non-uniformities in the respiratory volume versus time data occurring in synchrony with the applied pressure pulses and having a duration similar to the duration of the applied pressure pulses.

15. The computer-implemented method of claim 14 wherein the non-uniformities comprise abrupt reversals of airflow or abrupt increases in airflow.

16. The computer-implemented method of claim 14 wherein the non-uniformities are also searched for in time-differentiated respiratory volume.

17. The computer-implemented method of claim 12 further comprises determining respiratory resistance versus frequency data from the received data.

18. The computer-implemented method of claim 17 wherein deciding that a tongue position artifact (TPA) is present further comprises comparing the determined respiratory resistance versus frequency data with at least one set of previously-determined respiratory resistance versus frequency data.

19. The computer-implemented method of claim 12 further comprises determining respiratory resistance versus time data from the received data.

20. The computer-implemented method of claim 19 wherein deciding that a vocal cord adduction artifact (VCAE) is present further comprises comparing the determined respiratory resistance during inspiration with the determined respiratory resistance during expiration.

21. A computer system comprising:
a processor;
a memory operatively coupled to the processor; and
a communications interface linked directly or indirectly to at least one FOT device,
wherein the memory comprises stored instructions for causing the processor to perform the methods of claim 20.

22. The computer system of claim 21 further comprising an FOT device linked to the communications interface.

23. A method for assessing the pulmonary status of a plurality of subjects comprising:
receiving data from forced oscillation technique (FOT) measurements from a plurality of subjects, or from a whole body plethysmographic (WBP) measurements from a plurality of subjects;
obtaining results from the received data including one of more of an integrative measure characterizing specific airway resistance and an indication of whether or not artifact or distortion is present or absent in the received data; and
outputting the results for the plurality of subjects.

24. A method for screening for the effects of a chemical or pharmaceutical agent comprising:
receiving data from forced oscillation technique (FOT) measurements on at least one subject, or from a whole body plethysmographic (WBP) measurements of at least one subject, wherein the agent has not been administered to the one or more subjects;
obtaining first results from the received data including one of more of an integrative measure characterizing specific airway resistance and an indication of whether or not artifact or distortion is present or absent in the received data;
administering the agent to the one or more subjects;
receiving data from forced oscillation technique (FOT) measurements on at least one subject, or from a whole body plethysmographic (WBP) measurements of at least one subject, wherein the agent has been administered to the one or more subjects;
obtaining second results from the received data including one of more of an integrative measure characterizing specific airway resistance and an indication of whether or not artifact or distortion is present or absent in the received data; and
comparing the first and the second results.

25. A computer-implemented method of processing respiratory data comprising:
receiving respiratory data characterizing at least respiratory pressure and respiratory airflow throughout one or more cycles of respiration;
deriving respiratory resistance versus frequency from the received data; and
determining that a tongue position artifact (TPA) is present by comparing the determined respiratory resistance versus frequency data with at least one set of previously-determined respiratory resistance versus frequency data.

26. The computer-implemented method of claim 25 wherein a TPA is determined to be present if the determined respiratory resistance versus frequency data exceeds the previously-determined respiratory resistance versus frequency data by a substantially constant amount over a frequency range greater the 5 Hz.

27. The computer-implemented method of claim 26 where an amount is substantially constant if is varies by no more than 20-25%.

28. The computer-implemented method of claim 25 further comprising determining the received respiratory data by an respiratory airflow perturbation technique.

29. The computer-implemented method of claim 28 wherein the respiratory airflow perturbation technique comprises one or more a forced oscillation technique or use of an airflow perturbation device.

30. A computer-implemented method of processing respiratory data comprising:
receiving respiratory data characterizing at least respiratory pressure and respiratory airflow throughout one or more cycles of respiration;
deriving respiratory resistance and tidal volume versus time from the received data; and
determining that a vocal cord adduction artifact (VCAE) is present by comparing the determined airway resistance during inspiration with the determined airway resistance during expiration.

31. The computer-implemented method of claim 30 wherein a VCAE artifact is determined to be present if airway resistance during expiration exceeds airway resistance during inspiration, and if airway resistance during the course of expiration increases, fails to decrease, or substantially slows its rate of decrease during the latter 60% of expiration.

32. The computer-implemented method of claim 30 wherein a VCAE artifact is determined to be present if the maximum of airway resistance lags in time the maximum of the tidal volume.

33. The computer-implemented method of claim 30 further comprising:
determining the respiratory reactance versus time from the received data; and
determining that an expiratory flow limitation (EFL) is present by comparing the determined airway reactance during inspiration with the determined airway reactance during expiration.

34. The computer-implemented method of claim 33 wherein an EFL is determined to be present if airway reactance during expiration exceeds airway reactance during inspiration.

35. The computer-implemented method of claim 33 wherein an EFL is determined to be present if the maximum of airway reactance lags in time the maximum of the tidal volume.

36. The computer-implemented method of claim 30 further comprising determining the received respiratory data by an respiratory airflow perturbation technique.
